(19) United States
(12) Patent Application Publication
(10) Pub. No.: US 2023/0139056 A1
(43) Pub. Date: May 4, 2023

(54) METHODS, COMPOSITIONS AND REAGENTS FOR PREPARING COMPOUNDS

(71) Applicant: Regents of the University of Minnesota, Minneapolis, MN (US)

(72) Inventors: Ambika Bhagi-Damodaran, Minneapolis, MN (US); Sourav Chatterjee, Minneapolis, MN (US)

(73) Assignee: Regents of the University of Minnesota, Minneapolis, MN (US)

(21) Appl. No.: 17/976,580

(22) Filed: Oct. 28, 2022

Related U.S. Application Data
(60) Provisional application No. 63/273,674, filed on Oct. 29, 2021.

(51) Int. Cl. C12P 7/26; C12P 11/00; C12P 13/00; C12N 9/02

(52) U.S. Cl. CPC C12P 7/26; C12P 11/00; C12P 13/00; C12N 9/0071

(57) ABSTRACT

Disclosed herein are reactions, methods, reagents and compositions that utilize a nonheme iron halogenase enzyme to prepare compounds.

### CROSS REFERENCE TO RELATED APPLICATION

This application claims priority to U.S. Provisional Application Number 63/273,674 that was filed on Oct. 29, 2021. The entire content of the application referenced above is hereby incorporated by reference herein.

### GOVERNMENT FUNDING

This invention was made with government support under CBET-2046527 awarded by the National Science Foundation. The government has certain rights in the invention.

### BACKGROUND OF THE INVENTION

New methods, synthetic reactions, reagents and compositions to prepare compounds are highly sought. One reaction of particular importance is the substitution of a hydrogen bonded to a carbon (e.g., hydrogen bonded to a carbon by a single bond wherein the carbon is sp³ hybridized). Such reactions have very significant utility in making new compounds and making known compounds in a more efficient manner.

Accordingly, there is an ongoing need to develop new reactions or methods or compositions or reaction reagents (e.g., ligand activators) to make compounds (e.g., compounds wherein a hydrogen bonded to a carbon is substituted with another atom or group) Such new reactions or methods or compositions or reaction reagents (e.g., ligand activators) may also provide one or more advantages such as improved reaction selectivity or lower cost materials (e.g., substrates or reagents), fewer waste products or less hazardous waste products or require less severe reaction conditions such as lower temperature or pressure.

### SUMMARY OF THE INVENTION

Reactions, methods, reagents, and compositions disclosed herein can be used to prepare compounds including new compounds and known compounds.

Accordingly, one embodiment provides a method to convert a —CH- group of a substrate compound or a salt thereof, to a corresponding —CX- group of a product compound or a salt thereof, comprising contacting the substrate compound or a salt thereof with a nonheme iron halogenase enzyme and a ligand activator, wherein:

the carbon atom of the —CH- group and the —CX- group is sp³ hybridized;

X is halo, —N₃, —NO₂, —CN, or —SR; and

R is H or (C₁-C₈)alkyl.

### BRIEF DESCRIPTION OF THE FIGURES

FIGS. 1A-1B show the structure of NH—Fe Hals and the reaction. FIG. 1A shows the macromolecular and active site structure of SyrB2 halogenase. FIG. 1B shows the natural reaction of SyrB2 halogenase.

FIG. 2 shows C—H Functionalization reaction of NHFe-Hal with ligand activators. The substrate is a Thr amino acid bound to another protein. The substrates will change based on the NHFe-Hal being used. X is halo, N₃, NO₂, CN, or SR.

FIGS. 3A-3B show assays demonstrating C—H fluorination via ligators. FIG. 3A shows that UPLC-MS/MS is an effective method to determine amino acid concentrations in nM to mM range. The amino acids and their derivatives are tagged with a chromophore AQC and then subjected to UPLC-MS/MS analysis. FIG. 3B shows UPLC-MS/MS results of the detection of Thr (substrate), Thr-OH (side-product), and Thr-F (main product).

FIG. 4 shows the matrix-assisted laser desorption ionization—time of flight (MALDI-TOF) mass spectra for proteins used in Example 1. Expected masses are as follows: SyrB1-68.3 kDa, SyrB2-37.5 kDa, Sfp-27.2 kDa, TycF-33.4 kDa.

FIG. 5 shows a proposed reaction mechanism and catalytic cycle for C—H substitution reaction by halogenase enzyme complex with ligand activator (LIG). It is to be understood that the invention is in not limited in any manner by the proposed reaction mechanism.

Structure 1: The ligand activator (LIG) is required for the halogenase enzyme to bind to the non-heme iron wherein the carboxylate and keto groups of the ligand activator coordinate to the iron. The ligand activator also tunes the electron density of the iron atom so it can bind the anion of interest (X⁻). For example, electron donating ligand activators bind to electron withdrawing fluoride.

(1)→(2): The substrate (R—H) docks in close to the active site of halogenase and the water molecule bound to iron is removed.

(2)→(3): Oxygen binds to the iron and abstracts an electron from it forming an Fe(III)-superoxo intermediate.

(3)→(4): The carboxylate group of the ligand activator reacts with the superoxo, loses its iron binding carboxylate as carbon dioxide and enables the activation of non-heme iron to oxo-ferryl in structure (4).

(4)→(5): The oxo-ferryl intermediate is able to extract an H radical from the substrate's C—H bond that enables C radical formation (R′).

(5)→(6): The anion (X) bound to non-heme iron rebounds to the radical carbon and forms the C—X bond. (6)→(1): The iron in the halogenase is able to take protons, a new ligand activator, substrate, and anion from the solution to undergo further turnovers starting from structure 1 again.

FIG. 6 shows the chlorination reaction of Thr using SyrB2 using no ligand activators, 20G (2-oxo glutarate), and designed ligand activator 11. In the presence of 20G and 11, >85% of Thr is chlorinated as determined by UPLC-MS/MS. For each bar graph, the left bar is Cl-Thr, the middle bar graph HO-Thr, and the right bar is Thr.

### DETAILED DESCRIPTION

Chemo-, regio- and stereo-selective substitution of C(sp³)-H bonds is a reaction that is highly sought and of great importance in chemistry. Conducting such reactions in non-harsh conditions of temperature or pressure or using non-precious metals and/or reagents to conduct the reactions is an even greater challenge. Enzymes and specifically metalloenzymes are known to facilitate this type of transformation in biological systems.

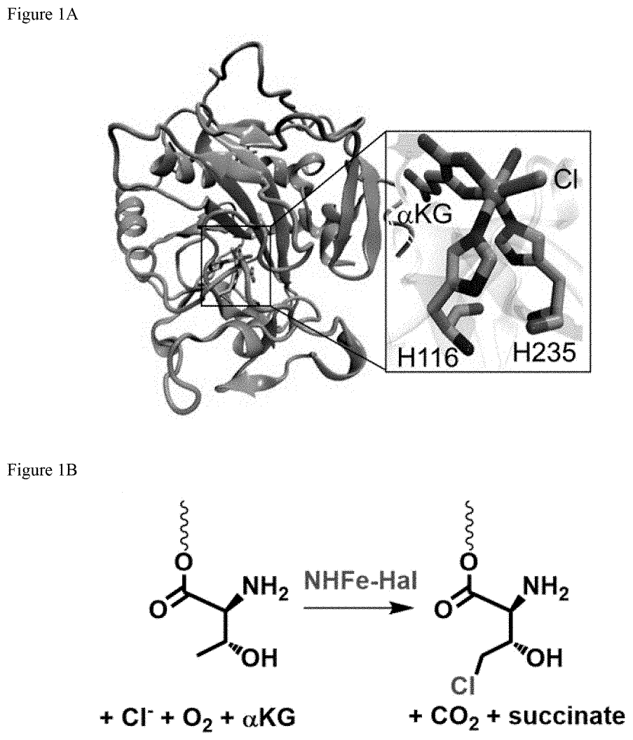

Described herein are reactions that utilize non-heme iron halogenases (NH-Fe Hals) that contain a non-heme iron bound to the protein through histidine residues, an alpha-ketoglutarate (aKG), and a chloride molecule (FIG. 1A). The NH-Fe Hals are activated by O₂ to a halo-ferryl species which is capable of abstracting an H radical from the C(sp³)-H substrate and transforming it to a C—Cl bond (FIG. 1B). Overall, by using inexpensive reagents like halide, oxygen, iron, and aKG, NH—Fe Hals are able to convert C—H bonds to C—Cl bonds in specific substrates. Numerous NH—Fe Hals have been reported in the last 20 years, including SyrB2, KthP, CytC3, BarB1/BarB2, CurA, CmaB, HctB, WelO5, AmbO5, AdeV, BesD, HalB, HalC, HalD, and HalE. These enzymes have been shown to facilitate reactions involving a variety of amino acid, nucleotides, and other natural product substrates.

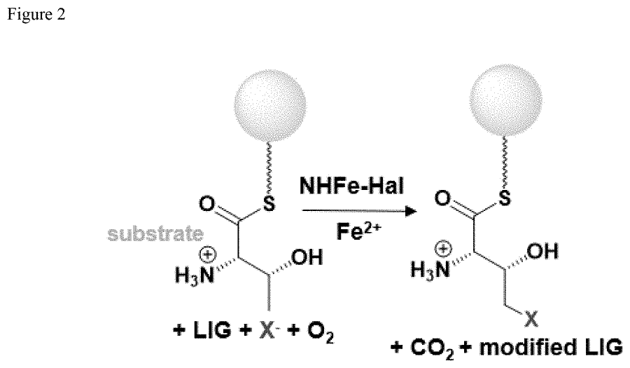

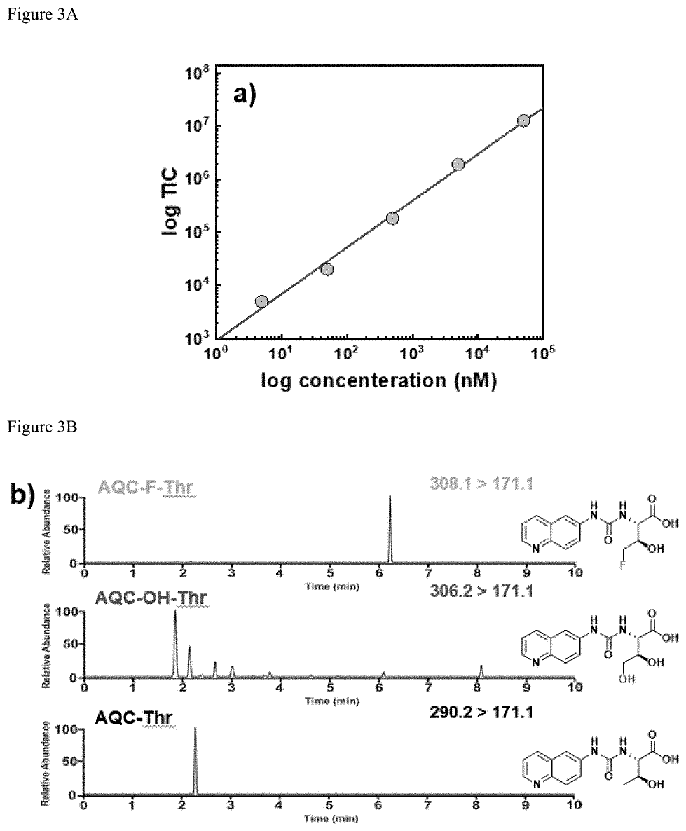

As described herein, aKG analogous ligand activators (also called “ligators” herein) are utilized as a means to broaden the reactivity profile of NH—Fe Hals and engineer the NH—Fe Hals to perform C—H substitution to C—X (where X=F, I, Br, N₃, NO₂, CN, SH, FIG. 2). The ligators tune electron density of non-heme iron in the active site of NH—Fe Hal enzymes, such that they are able to bind various anions such as F⁻, I⁻, Br⁻, N₃⁻, NO₂⁻, CN⁻, ⁻SH. For instance, computational, UV-Vis, Mössbauer spectroscopic, and LC-MS/MS assay results have shown the electron donating N-oxalylglycine (NOG) ligator enables iron to bind and activate electron withdrawing fluoride enabling fluorination of Thr amino acid by SyrB2 halogenase (FIG. 3). Please see the methods section of Example 1 for details on expression, purification of SyrB2 halogenase and the fluorination assay. In some of the reaction conditions with ligator NOG, little to no side products (hydroxylated substrates) are observed. The ligators enable efficient catalysis of non-activated C—H bond fluorination with inexpensive starting materials and under non-oxidative conditions. The starting materials used are inexpensive and non-hazardous such as sodium fluoride, iron containing mohr's salt, and oxygen. The nonheme iron halogenases that can enable selective C—H fluorination and other substitutions with the designed ligators include KthP, CytC3, BarB1/BarB2, CurA, CmaB, HctB, WelO5, AmbO5, AdeV, BesD, HalB, HalC, HalD, and HalE. Additionally, the methods can be used with various fluoride sources like sodium fluoride, potassium fluoride, tetrabutylammonium fluoride (TBAF), tetrabutylammonium fluoride +AgF/CsF, diethylaminosulfur trifluoride (DAST), (diethylamino)difluorosulfonium tetrafluoroborate (XtalFluor-E), N-Fluoro-N′-methyl-triethylenediamine bis(tetrafluoroborate) (Selectfluor II) and N-Fluorobenzenesulfonimide (NFSI).

The following definitions are used, unless otherwise described: halo or halogen is fluoro, chloro, bromo, or iodo. Alkyl and alkoxy, etc. denote both straight and branched groups but reference to an individual radical such as propyl embraces only the straight chain radical (a branched chain isomer such as isopropyl being specifically referred to).

The term “(Cₐ-C_{b})alkyl” wherein a and b are integers refers to a straight or branched chain hydrocarbon alkyl radical having from a to b carbon atoms. Thus, when a is 1 and b is 6, for example, the term includes methyl, ethyl, n-propyl, isopropyl, n-butyl, isobutyl, sec-butyl, t-butyl, n-pentyl and n-hexyl.

The term “heteroalkyl” refers to a straight or branched chain hydrocarbon alkyl radical, consisting of the stated number of carbon atoms and from one to three heteroatoms selected from the group consisting of O, N, and S, wherein the nitrogen and sulfur atoms can optionally be oxidized and the nitrogen heteroatom can optionally be quaternized. The heteroatom(s) O, N and S can be placed at any interior position of the heteroalkyl group. Examples include —CH₂—CH₂—O—CH₃, —CH₂—CH₂—NH—CH₃, —CH₂—CH₂—N(CH₃)—CH₃, —CH₂—S—CH₂—CH₃, —S(O)—CH₃, and —CH₂—CH₂—S(O)₂—CH₃

The term “alkenyl” refers to an unsaturated alkyl radical having one or more double bonds. Examples of such alkenyl groups include ethenyl, 1- and 3-propenyl, 3-butenyl, and higher homologs and isomers. It is to be understood that the alkenyl can be branched or unbranched.

The term “alkynyl” refers to an unsaturated alkyl radical having one or more triple bonds. Examples of such alkynyl groups include ethynyl, 1- and 3-propynyl, 3-butynyl, and higher homologs and isomers. It is to be understood that the alkynyl can be branched or unbranched.

The term “aryl” as used herein refers to a single aromatic ring or a multiple condensed ring system wherein the ring atoms are carbon. For example, an aryl group can have 6 to 10 carbon atoms, or 6 to 12 carbon atoms. Aryl includes a phenyl radical. Aryl also includes multiple condensed ring systems (e.g., ring systems comprising 2 rings) having about 9 to 12 carbon atoms or 9 to 10 carbon atoms in which at least one ring is aromatic. Such multiple condensed ring systems may be optionally substituted with one or more (e.g., 1 or 2) oxo groups on any cycloalkyl portion of the multiple condensed ring system. It is to be understood that the point of attachment of a multiple condensed ring system, as defined above, can be at any position of the ring system including an aryl or a cycloalkyl portion of the ring. Typical aryl groups include, but are not limited to, phenyl, indenyl, naphthyl, 1, 2, 3, 4-tetrahydronaphthyl, anthracenyl, and the like. In embodiment aryl is phenyl or naphthyl.

The term “heteroaryl” as used herein refers to a single aromatic ring or a multiple condensed ring system. The term includes single aromatic rings from about 1 to 6 carbon atoms and about 1-4 heteroatoms selected from the group consisting of oxygen, nitrogen and sulfur in the rings. The sulfur and nitrogen atoms may also be present in an oxidized form provided the ring is aromatic. Such rings include but are not limited to pyridyl, pyrimidinyl, oxazolyl or furyl. The term also includes multiple condensed ring systems (e.g. ring systems comprising 2 rings) wherein a heteroaryl group, as defined above, can be condensed with one or more heteroaryls (e.g., naphthyridinyl), heterocycles, (e.g., 1, 2, 3, 4-tetrahydronaphthyridinyl), cycloalkyls (e.g., 5,6,7,8-tetrahydroquinolyl) or aryls (e.g. indazolyl) to form a multiple condensed ring system. Such multiple condensed ring systems may be optionally substituted with one or more (e.g., 1 or 2) oxo groups on the cycloalkyl or heterocycle portions of the condensed ring. In one embodiment a monocyclic or bicyclic heteroaryl has 5 to 10 ring atoms comprising 1 to 9 carbon atoms and 1 to 4 heteroatoms. It is to be understood that the point of attachment of a multiple condensed ring system (as defined above for a heteroaryl) can be at any position of the multiple condensed ring system including a heteroaryl, heterocycle, aryl or cycloalkyl portion of the multiple condensed ring system and at any suitable atom of the multiple condensed ring system including a carbon atom and heteroatom (e.g., a nitrogen). Exemplary heteroaryls include but are not limited to pyridyl, pyrrolyl, pyrazinyl, pyrimidinyl, pyridazinyl, pyrazolyl, thienyl, indolyl, imidazolyl, oxazolyl, thiazolyl, furyl, oxadiazolyl, thiadiazolyl, quinolyl, isoquinolyl, benzothiazolyl, benzoxazolyl, indazolyl, quinoxalyl, quinazolyl, 5,6,7,8-tetrahydroisoquinolinyl, benzofuranyl, benzimidazolyl and thianaphthenyl.

The term “heterocyclyl” or “heterocycle” as used herein refers to a single saturated or partially unsaturated ring or a multiple condensed ring system. The term includes single saturated or partially unsaturated rings (e.g., 3, 4, 5, 6 or 7-membered rings) from about 1 to 6 carbon atoms and from about 1 to 3 heteroatoms selected from the group consisting of oxygen, nitrogen and sulfur in the ring. The ring may be substituted with one or more (e.g., 1, 2 or 3) oxo groups and the sulfur and nitrogen atoms may also be present in their oxidized forms. Such rings include but are not limited to azetidinyl, tetrahydrofuranyl or piperidinyl. It is to be understood that the point of attachment for a heterocycle can be at any suitable atom of the heterocycle. Exemplary heterocycles include, but are not limited to aziridinyl, azetidinyl, pyrrolidinyl, piperidinyl, homopiperidinyl, morpholinyl, thiomorpholinyl, piperazinyl, tetrahydrofuranyl, dihydrooxazolyl, tetrahydropyranyl and tetrahydrothiopyranyl.

The term “haloalkyl” includes an alkyl group as defined herein that is substituted with one or more (e.g., 1, 2, 3, or 4) halo groups. One specific halo alkyl is a “(C₁-C₆)haloalkyl”.

The term cycloalkyl, carbocycle, or carbocyclyl includes saturated and partially unsaturated carbocyclic ring systems. In one embodiment the carbocyclyl is a monocyclic carbocyclic ring. Such carbocyclyl include “(C₃-C₇)carbocyclyl” and “(C₃-C₈)cycloalkyl”.

Certain embodiments of the invention are provided herein. It is to be understood that two or more embodiments may be combined.

Specific values listed below for radicals, substituents, and ranges, are for illustration only; they do not exclude other defined values or other values within defined ranges for the radicals and substituents.

Specifically, (C₁-C₆)alkyl can be methyl, ethyl, propyl, isopropyl, butyl, iso-butyl, sec-butyl, pentyl, 3-pentyl, or hexyl; (C₁-C₆)alkoxy can be methoxy, ethoxy, propoxy, isopropoxy, butoxy, iso-butoxy, sec-butoxy, pentoxy, 3-pentoxy, or hexyloxy; (C₃-C₈)cycloalkyl can be cyclopropyl, cyclobutyl, cyclopentyl, or cyclohexyl; (C₁-C₆)haloalkyl can be iodomethyl, bromomethyl, chloromethyl, fluoromethyl, trifluoromethyl, 2-chloroethyl, 2-fluoroethyl, 2,2,2-trifluoroethyl, or pentafluoroethyl; aryl can be phenyl, indenyl, or naphthyl; and heteroaryl can be furyl, imidazolyl, triazolyl, triazinyl, oxazolyl, isoxazolyl, thiazolyl, isothiazoyl, pyrazolyl, pyrrolyl, pyrazinyl, tetrazolyl, pyridyl, (or its N-oxide), thienyl, pyrimidinyl (or its N-oxide), indolyl, isoquinolyl (or its N-oxide) or quinolyl (or its N-oxide).

### Nonheme Iron Halogenase

The term nonheme iron halogenase as used herein includes any nonheme iron halogenase that enables the C—H substitutions including selective reactions described herein when used in combination with ligand activators. In one embodiment the nonheme iron halogenase is selected from the group consisting of:
SyrB2 (Blasiak, L. C., et al., . Nature 440,368-371 (2006); Vaillancourt, F. H., et al., Proc. Natl. Acad. Sci. 102,10111-10116 (2005); Matthews, M. L. et al., Proc. Natl. Acad. Sci. U. S. A. 106,17723-17728 (2009));
KthP (Jiang, W. et al., Biochemistry 50,6063-6072 (2011));
CytC3 (Wong, C., et al., J. Am. Chem. Soc. 131,4872-4879 (2009));
BarB 1/BarB2 (Galonić, D. P., et al., J. Am. Chem. Soc. 128,3900-3901 (2006));
CurA (Khare, D. et al. Proc. Natl. Acad. Sci. U. S. A. 107,14099-14104 (2010));
CmaB (Vaillancourt, F. H., et al., Nature 436,1191-1194 (2005));
HctB (Pratter, S. M. et al., ChemBioChem 15,567-574 (2014));
WelO5 (PubMed. https://pubmed.ncbi.nlm.nih.gov/27348090/);
AmbO5 (Hillwig, M. L., et al., Angew. Chem. Int. Ed Engl. 55,5780-5784 (2016));
AdeV (Zhao, C. et al., Angew. Chem. 132,9565-9571 (2020));
BesD (Neugebauer, M. E. et al., Nat. Chem. Biol. 15,1009-1016 (2019));
HalB, (Neugebauer, M. E. et al., Nat. Chem. Biol. 15,1009-1016 (2019));
HalC, (Neugebauer, M. E. et al., Nat. Chem. Biol. 15,1009-1016 (2019));
HalD, (Neugebauer, M. E. et al., Nat. Chem. Biol. 15,1009-1016 (2019)); and
HalE (Neugebauer, M. E. et al., Nat. Chem. Biol. 15,1009-1016 (2019)).

### Ligand Activator

Ther term ligand activator or ligator as used herein is an aKG analogue that binds (or ligates) to iron in nonheme iron halogenase and activates the iron molecule to react with oxygen and perform the C—H substitution reaction.

In one embodiment the ligand activator comprises two or more oxygen atoms, wherein at least two of the oxygen atoms coordinate to the iron of the nonheme iron enzyme.

In one embodiment the ligand activator comprises one or more oxo (═O) groups.

In one embodiment the ligand activator comprises two or more oxo (═O) groups.

In one embodiment the ligand activator comprises one or more hydroxy groups.

In one embodiment the ligand activator comprises two oxo (═O) groups and one hydroxy group.

In one embodiment the ligand activator includes two oxo (═O) groups and one hydroxy group.

In one embodiment the ligand activator comprises one or more groups of formula IV:

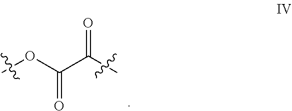

In one embodiment the ligand activator includes one or more groups of formula IV:

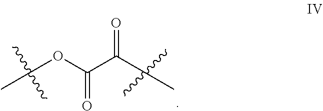 [SMILES: *OC(=O)C(*)=O]

In one embodiment the ligand activator comprises one or more groups of formula IV:

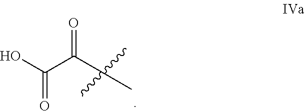 [SMILES: *C(=O)C(=O)O]

In one embodiment the ligand activator includes one or more groups of formula IV:

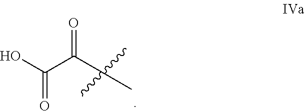 [SMILES: *C(=O)C(=O)O]

In one embodiment the ligand activator comprises a substituted or unsubstituted compound of formula Ia, formula IIa or formula IIIa:

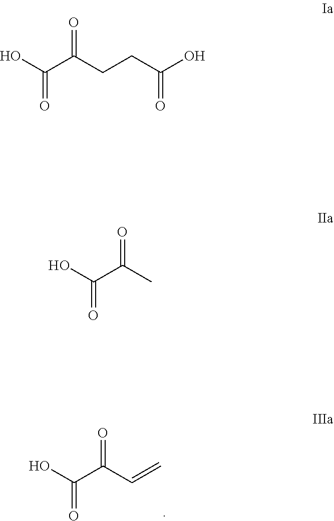

One embodiment provides a compound (i.e., a ligand activator) as described in any of the following embodiments. It is to be understood that two or more embodiments (embodiments directed to the ligand activators of formulas I, II, or III as well as the embodiments directed to any of the variables describing these ligand activators) may be combined.

In one embodiment the ligand activator is a compound of formula I, formula II or formula III:

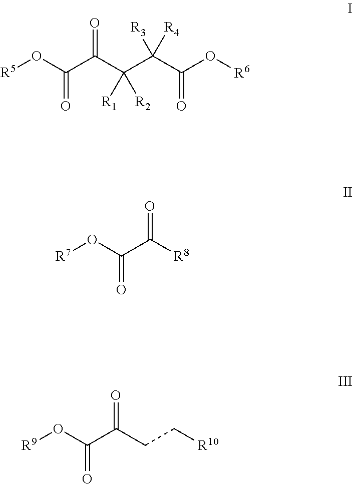

wherein:

the dashed bond of the compound of formula III is a single bond or a double bond;

R¹, R², R³, R⁴, are each independently hydrogen, halo, (C₁-C₈)alkyl, —CO₂H, —OH, 5-6 membered heteroaryl, 4-7 membered heterocyclyl, or phenyl, wherein the (C₁-C₈)alkyl, 5-6 membered heteroaryl, 4-7 membered heterocyclyl, or phenyl is optionally substituted with one or more groups independently selected from halo, oxo, —CN, —NO₂, —CO₂H, —OH, (C₁-C₆)alkyl, (C₁-C₆)haloalkyl, —O (C₁-C₆)alkyl, —O (C₁-C₆)haloalkyl and SO₂R^{a};

R⁵ is hydrogen or (C₁-C₈)alkyl;

R⁶ is hydrogen, (C₁-C₈)alkyl, or phenyl, wherein phenyl is optionally substituted with one or more groups independently selected from halo, oxo, —CN, —NO₂, —CO₂H, —OH, (C₁-C₆)alkyl, (C₁-C₆)haloalkyl, —O (C₁-C₆)alkyl, —O (C₁-C₆)haloalkyl and SO₂R^{a};

R⁷ is hydrogen or (C₁-C₈)alkyl;

R⁸ is (C₃-C₇)carbocyclyl, 4-7 membered heterocyclyl, phenyl, OR^{b}, SR^{b}, -(C₁-C₆)alkylOR^{b}, or -(C₁-C₆)alkylSR^{b} wherein the (C₃-C₇)carbocyclyl, 4-7 membered heterocyclyl, phenyl, -(C₁-C₆)alkylOR^{b}, or -(C₁-C₆)alkylSR^{b}, is optionally substituted with one or more groups independently selected from halo, oxo, —CN, —NO₂, —CO₂H, —OH, (C₁-C₆)alkyl, (C₁-C₆)haloalkyl, —CO₂H, —O (C₁-C₆)alkyl, —O (C₁-C₆)haloalkyl and SO₂R^{a};

R⁹ is hydrogen or (C₁-C₈)alkyl;

R¹⁰ is —(C₁-C₁₀)alkyl, —(C₁-C₁₀)alkenyl, R^{10a}, -(C₁-C₁₀)alkylR^{10a}, or -(C₁-C₁₀)alkenylR¹⁰a, wherein the -(C₁-C₁₀)alkyl, -(C₁-C₁₀)alkenyl, -(C₁-C₁₀)alkylR^{10a}, or -(C₁-C₁₀)alkenylR^{10a} is optionally substituted with one or more halo;

each R^{11a} is independently aryl or heteroaryl, wherein the aryl or heteroaryl is optionally substituted with one or more groups independently selected from halo, —CN, —NO₂, —CO₂H, —OH, (C₁-C₆)alkyl, (C₁-C₆)haloalkyl, —O (C₁-C₆)alkyl, —O (C₁-C₆)haloalkyl, and SO₂R^{a};

R^{a} is —OH, (C₁-C₆)alkyl, or (C₁-C₆)haloalkyl;

R^{b} and RC are each independently (C₁-C₈)alky, wherein (C₁-C₈)alky is optionally substituted with one or more groups independently selected from halo, CO₂H, oxo, —OH, 5-6-membered heteroaryl and —N═C(NR^{d}₂)₂, wherein 5-6-membered heteroaryl is optionally substituted with one or more groups independently selected from halo, oxo, —CN, —NO₂, —CO₂H, —OH, (C₁-C₆)alkyl, (C₁-C₆)haloalkyl, —O (C₁-C₆)alkyl, —O (C₁-C₆)haloalkyl, and SO₂R^{a};

each R^{d} is independently hydrogen or (C₁-C₆)alky;

or a salt thereof.

In one embodiment the ligand activator is a compound of formula I, formula II or formula III:

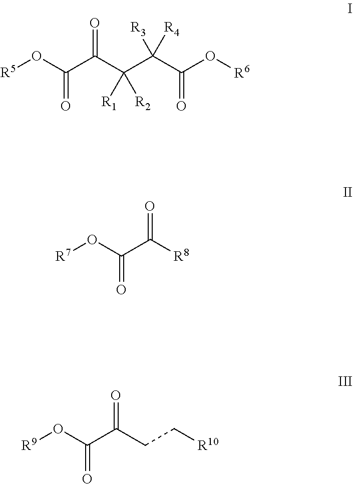

wherein:

the dashed bond of the compound of formula III is a single bond or a double bond;

R¹, R², R³, R⁴, are each independently hydrogen, halo, (C₁-C₈)alkyl, —CO₂H, —OH, 5-6 membered heteroaryl, 4-7 membered heterocyclyl, or phenyl, wherein the (C₁-C₈)alkyl, 5-6 membered heteroaryl, 4-7 membered heterocyclyl, or phenyl is optionally substituted with one or more groups independently selected from halo, oxo, —CN, —NO₂, —CO₂H, —OH, (C₁-C₆)alkyl, (C₁-C₆)haloalkyl, —O (C₁-C₆)alkyl, —O (C₁-C₆)haloalkyl and SO₂R^{a};

R⁵ is hydrogen or (C₁-C₈)alkyl;

R⁶ is hydrogen, (C₁-C₈)alkyl, or phenyl, wherein phenyl is optionally substituted with one or more groups independently selected from halo, oxo, —CN, —NO₂, —CO₂H, —OH, (C₁-C₆)alkyl, (C₁-C₆)haloalkyl, —O (C₁-C₆)alkyl, —O (C₁-C₆)haloalkyl and SO₂R^{a};

R⁷ is hydrogen or (C₁-C₈)alkyl;

R⁸ is (C₃-C₇)carbocyclyl, 4-7 membered heterocyclyl, phenyl, OR^{b}, SR^{b}, -(C₁-C₆)alkylOR^{b}, or -(C₁-C₆)alkylSR^{b} wherein the (C₃-C₇)carbocyclyl, 4-7 membered heterocyclyl, phenyl, -(C₁-C₆)alkylOR^{b}, or -(C₁-C₆)alkylSR^{b}, is optionally substituted with one or more groups independently selected from halo, oxo, —CN, —NO₂, —CO₂H, —OH, (C₁-C₆)alkyl, (C₁-C₆)haloalkyl, —CO₂H, —O (C₁-C₆)alkyl, —O (C₁-C₆)haloalkyl and SO₂R^{a};

R⁹ is hydrogen or (C₁-C₈)alkyl;

R¹⁰ is -(C₁-C₁₀)alkenyl, R^{10a}, -(C₁-C₁₀)alkylR^{10a}, or -(C₁-C₁₀)alkenylR¹⁰a, wherein the -(C₁-C₁₀)alkyl, -(C₁-C₁₀)alkenyl, -(C₁-C₁₀)alkylR^{10a}, or -(C₁-C₁₀)alkenylR^{10a} is optionally substituted with one or more halo;

each R^{10a} is independently phenyl, wherein the phenyl is optionally substituted with one or more groups independently selected from halo, —CN, —NO₂, —CO₂H, —OH, (C₁-C₆)alkyl, (C₁-C₆)haloalkyl, —O (C₁-C₆)alkyl, —O (C₁-C₆)haloalkyl, and SO₂R^{a};

R^{a} is —OH, (C₁-C₆)alkyl, or (C₁-C₆)haloalkyl;

R^{b} and R^{c} are each independently (C₁-C₈)alky, wherein (C₁-C₈)alky is optionally substituted with one or more groups independently selected from halo, CO₂H, oxo, —OH, 5-6-membered heteroaryl and —N═C(NR^{d}₂)₂, wherein 5-6-membered heteroaryl is optionally substituted with one or more groups independently selected from halo, oxo, —CN, —NO₂, —CO₂H, —OH, (C₁-C₆)alkyl, (C₁-C₆)haloalkyl, —O (C₁-C₆)alkyl, —O (C₁-C₆)haloalkyl, and SO₂R^{a};

each R^{d} is independently hydrogen or (C₁-C₆)alky; or a salt thereof.

In one embodiment the ligand activator is:

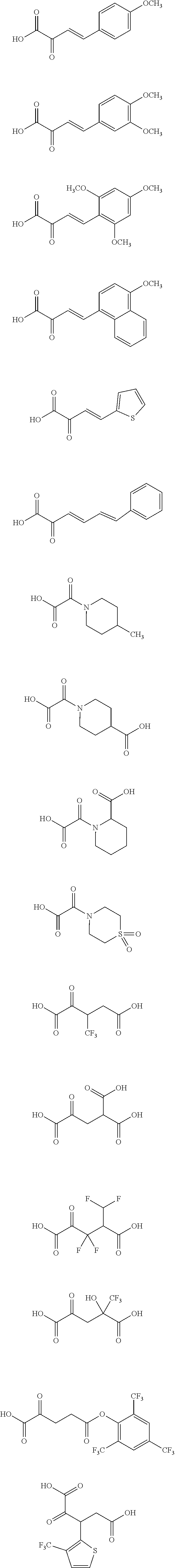

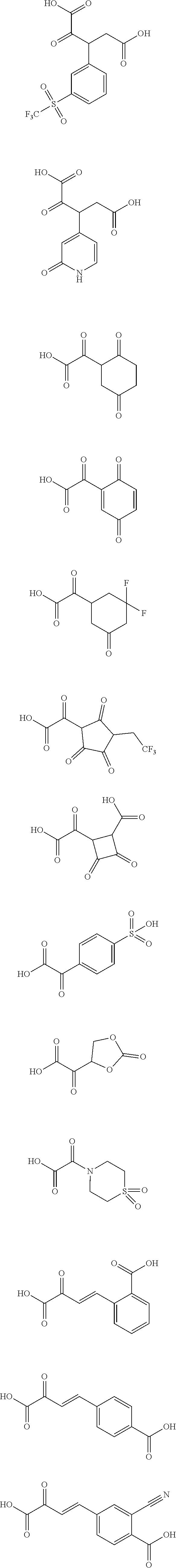

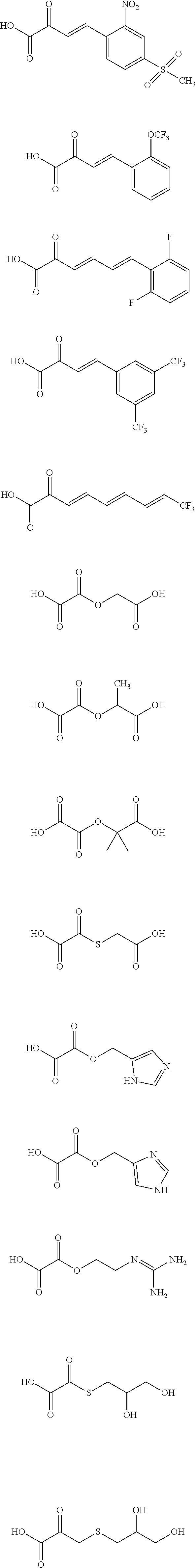

or an ester(s) or a salt thereof.

In one embodiment the ligand activator is:

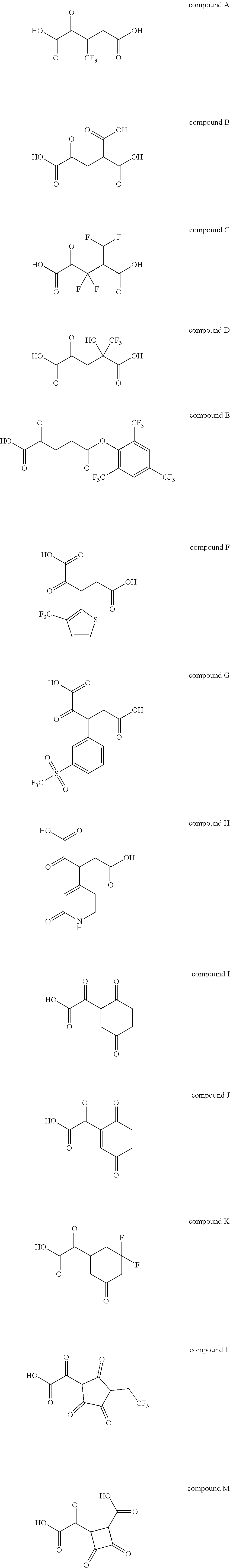

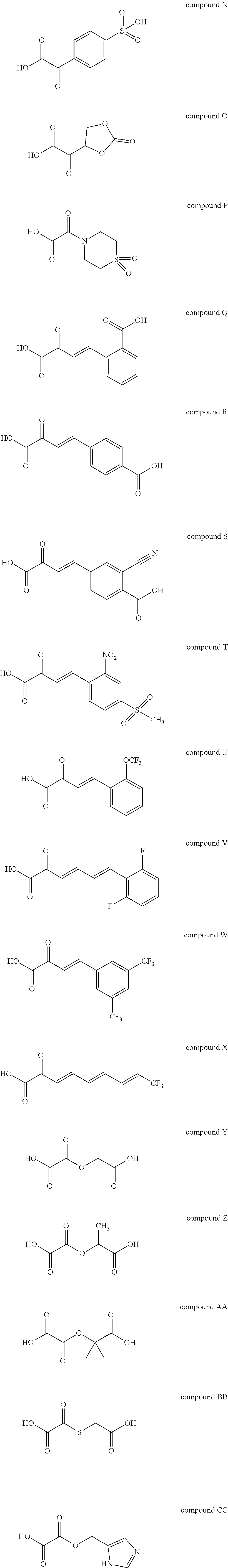

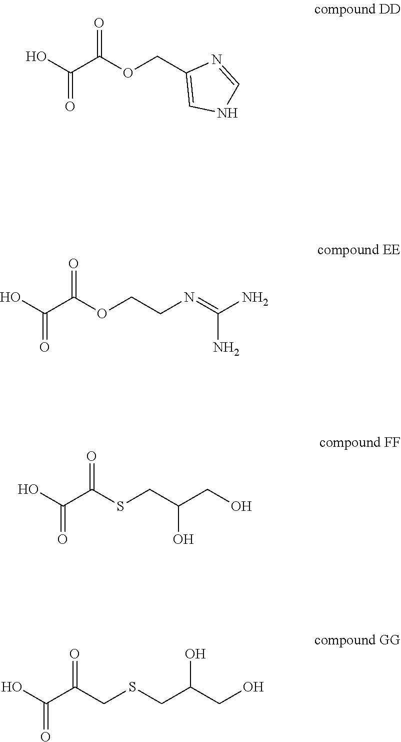 [SMILES: O=C(O)C(=O)OCc1c[nH]cn1 ; NC(N)=NCCOC(=O)C(=O)O ; O=C(O)C(=O)SCC(O)CO ; O=C(O)C(=O)CSCC(O)CO]

or an ester(s) or a salt thereof.

In one embodiment the ligand activator is:

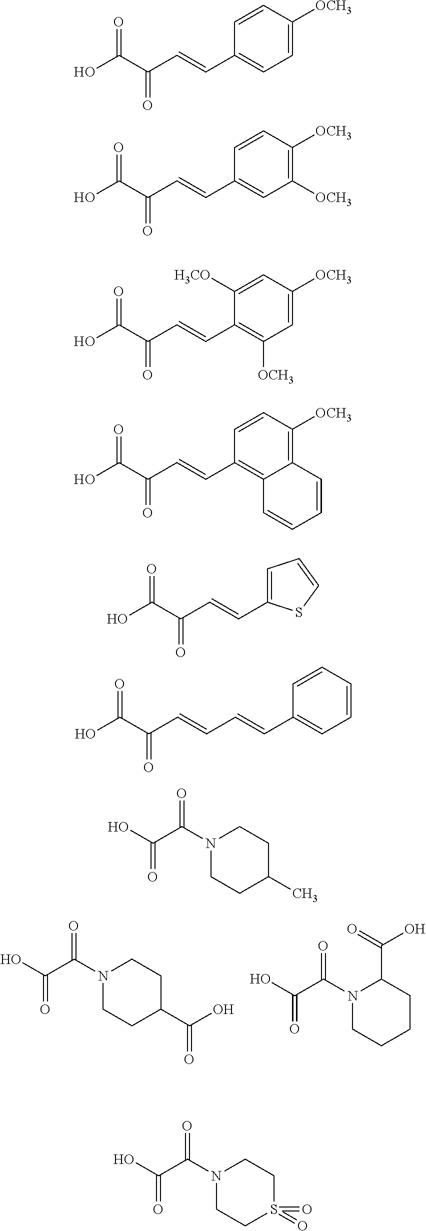

or an ester(s) or a salt thereof.

In one embodiment the ligand activator is a compound of formula III:

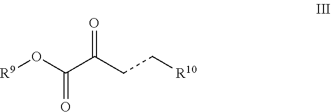

or a salt thereof.

In one embodiment the ligand activator is a compound of formula II:

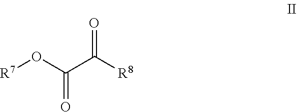

or a salt thereof.

In one embodiment the ligand activator is a compound of formula I:

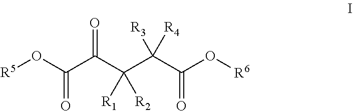

or a salt thereof.

In one embodiment R¹, R², R³, R⁴, are each independently hydrogen, (C₁-C₈)alkyl, —CO₂H, —OH, or 5-6 membered heteroaryl, wherein the (C₁-C₈)alkyl or 5-6 membered heteroaryl is optionally substituted with one or more groups independently selected from halo, oxo, —CN, —NO₂, —CO₂H, —OH, (C₁-C₆)alkyl, (C₁-C₆)haloalkyl, —O (C₁-C₆)alkyl, —O (C₁-C₆)haloalkyl and SO₂R^{a}.

In one embodiment R⁵ is hydrogen.

In one embodiment R⁶ is hydrogen or phenyl, wherein phenyl is optionally substituted with one or more groups independently selected from halo, oxo, —CN, —NO₂, —CO₂H, —OH, (C₁-C₆)alkyl, (C₁-C₆)haloalkyl, —O (C₁-C₆)alkyl, —O (C₁-C₆)haloalkyl and SO₂R^{a}.

In one embodiment R⁷ is hydrogen.

In one embodiment R⁸ is (C₃-C₇)carbocyclyl, 4-7 membered heterocyclyl, phenyl, OR^{b}, or SR^{b}, wherein the (C₃-C₇)carbocyclyl, 4-7 membered heterocyclyl, or phenyl is optionally substituted with one or more groups independently selected from halo, oxo, —CN, —NO₂, —CO₂H, —OH, (C₁-C₆)alkyl, (C₁-C₆)haloalkyl, —CO₂H, —O (C₁-C₆)alkyl, —O (C₁-C₆)haloalkyl and SO₂R^{a}.

In one embodiment R⁹ is hydrogen.

In one embodiment R¹⁰ is -(C₁-C₁₀)alkenyl, R^{10a}, or -(C₁-C₁₀)alkenylR^{10a}, wherein the -(C₁-C₁₀)alkenyl or -(C₁-C₁₀)alkenylR^{10a} is optionally substituted with one or more halo.

One embodiment provides a compound (i.e., a ligand activator) as described in any of the embodiments.

One embodiment provides a novel compound (i.e., a ligand activator) as described in any of the embodiments.

In one embodiment the ligand activator is 2-oxoglutaric acid or N-oxalylglycine or a salt thereof.

### Substrate Compound

The term substrate compound includes any compound that can bind to the nonheme iron halogenase enzyme and undergo the reactions described herein. The substrate compound includes at least one —CH- group wherein the carbon atom is sp^{a} hybridized (e.g., all bonds connected to the carbon atom are single bonds). In one embodiment the substrate compound is an amino acid, nucleotide, natural product or synthetic compound. In one embodiment the substrate compound is an amino acid, nucleotide, natural product or synthetic compound that has a molecular weight of less than 2000. In one embodiment the substrate compound is an amino acid, nucleotide, natural product or synthetic compound that has a molecular weight of less than 1500. In one embodiment the substrate compound is an amino acid, nucleotide, natural product or synthetic compound that has a molecular weight of less than 1000. In one embodiment the substrate compound is an amino acid, nucleotide, natural product or synthetic compound that has a molecular weight of less than 500. In one embodiment the substrate compound is an amino acid, nucleotide, natural product or synthetic compound that has a molecular weight of less than 250. In one embodiment the substrate compound is an amino acid, nucleotide, natural product or synthetic compound that has a molecular weight of between 10 and 250. In one embodiment the substrate compound is an amino acid, nucleotide, natural product or synthetic compound that has a molecular weight of between 10 and 500. In one embodiment the substrate compound is an amino acid, nucleotide, natural product or synthetic compound that has a molecular weight of between 10 and 1000. In one embodiment the substrate molecule includes one or more atoms selected from carbon, hydrogen, oxygen, nitrogen, phosphorus, sulfur and halogen. In one embodiment the substrate molecule includes one or more atoms selected from carbon, hydrogen, oxygen, nitrogen, and halogen. In one embodiment the substrate molecule includes one or more atoms selected from carbon, hydrogen, oxygen, nitrogen, phosphorous, and sulfur. In one embodiment the substrate molecule includes one or more atoms selected from carbon, hydrogen, oxygen, and nitrogen.

### Product Compound

The term product compound as used herein refers to the compound that is obtained after the reaction of the substrate compound. The product compound includes at least one —CX- group wherein the carbon atom is sp^{a} hybridized (e.g., all bonds connected to the carbon atom are single bonds). The X group can be any group that is obtained from the reactions described herein. In one embodiment X is halo, —N₃, —NO₂, —CN, or —SR, wherein R is H or (C₁-C₈)alkyl.

In one embodiment X is halo. In one embodiment X is F. In one embodiment X is Cl.

The invention will now be illustrated by the following non-limiting Example.

### EXAMPLE 1

### Methods and Materials:

### Chemicals

Yeast extract (Mol. Bio. grade), tryptone (Mol. Bio. grade), ethylenediaminetetraacetic acid disodium salt hydrate, sodium chloride, kanamycin, 4-(2-hydroxyethyl)-1-piperazineethanesulfonic acid (HEPES) (BioCertified), imidazole, coenzyme A salt hydrate, magnesium sulfate, disodium adenosine-5′-triphosphate (ATP), L-threonine, L-norvaline (arginase inhibitor), ferrous ammonium sulfate hexahydrate, Ellman's reagent (5,5′-dithio-bis nitrobenzoic acid (DTNB), BioReagent grade), 2-oxoglutaric acid sodium salt (20G), N-oxalylglycine (NOG, >99%, HPLC), sodium fluoride, sodium ascorbate, isopropanol (LC/MS grade), and sodium hydroxide were all purchased from Millipore-Sigma. Chemicals are of BioXtra grade unless otherwise stated. ¹³C₅-L-threonine was purchased from Cambridge Isotope Laboratories.

### General Procedure for the Synthesis of Compounds 7-12

To a pyruvic acid (646 mg, 520 7.34 mmol) solution in methanol (5 mL), methanolic sodium hydroxide (440 mg, 11 mmol in 2 mL methanol) was added dropwise at 0° C. After 30 min, a methanol solution of 4-methoxybenzaldehyde (1 g, 900 μL, 7.34 mmol in 1 mL methanol) was added dropwise as well. The solution slowly turned pale-yellow in color. The reaction mixture was further stirred at 10° C. for 24 h. The resulting precipitate was cooled down, filtered, and washed with ice cold methanol (3×2 mL) and then with diethyl ether (2×10 mL). The resulting sodium salt (compound 1) was isolated as pale-yellow solid (1.18 g, 71%). It was further taken into water (7.5 mL), stirred and 5% aqueous HCl was added under cold conditions until pH was ˜5-6. The corresponding acid was further extracted with 2% methanol in chloroform (2×20 mL). Combined organic layers were washed with cold water (5 mL), dried over anhydrous sodium sulfate, and evaporated to dryness under reduced pressure. The resulting α-keto acid compound 7 was isolated as a pale-yellow solid (984 mg, 65%).

Compounds 8-12 were prepared in a similar manner.

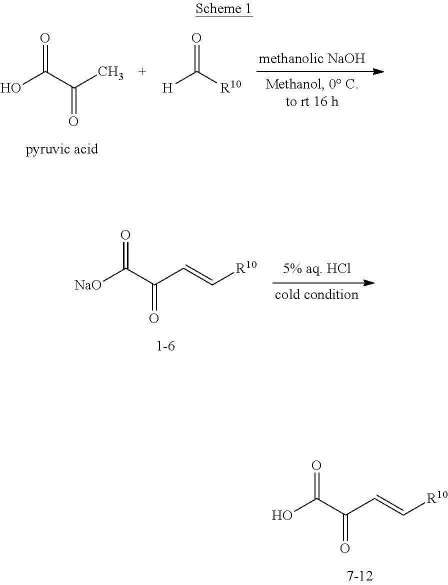

R¹⁰ is -(C₁-C₁₀)alkenyl, R^{10a}, -(C₁-C₁₀)alkylR^{10a}, or -(C₁-C₁₀)alkenylR^{10a}, wherein -(C₁-C₁₀)alkyl, —(C₁-C₁₀)alkenyl, -(C₁-C₁₀)alkylR^{10a}, or -(C₁-C₁₀)alkenylR^{10a} is optionally substituted with one or more halo; each

R^{10a} is independently aryl or heteroaryl, wherein the aryl or heteroaryl is optionally substituted with one or more groups independently selected from halo, —CN, —NO₂, —CO₂H, —OH, (C₁-C₆)alkyl, (C₁-C₆)haloalkyl, —O (C₁-C₆)alkyl, —O (C₁-C₆)haloalkyl, and SO₂R^{a}.

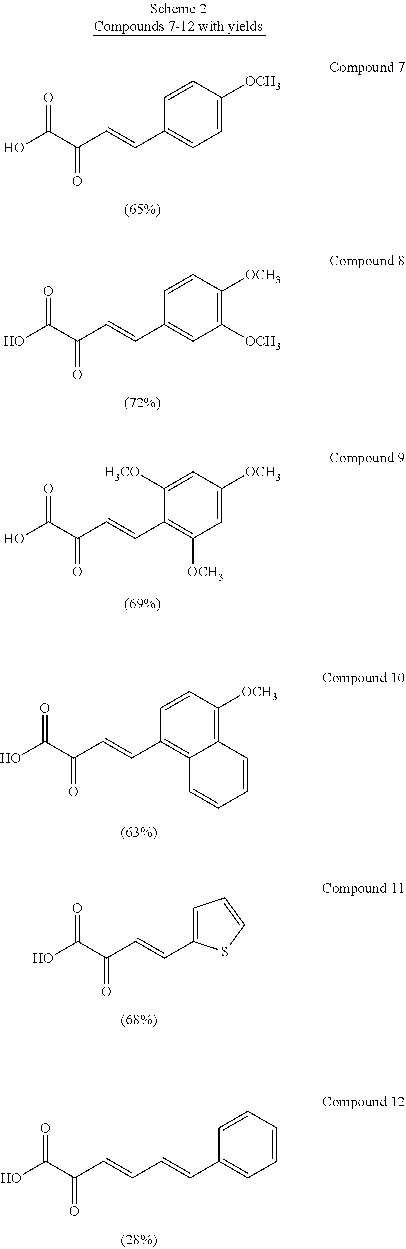 [SMILES: COc1ccc(/C=C/C(=O)C(=O)O)cc1 ; COc1ccc(/C=C/C(=O)C(=O)O)cc1OC ; COc1cc(OC)c(/C=C/C(=O)C(=O)O)c(OC)c1 ; COc1ccc(/C=C/C(=O)C(=O)O)c2ccccc12 ; O=C(O)C(=O)/C=C/c1cccs1 ; O=C(O)C(=O)/C=C/C=C/c1ccccc1]

### General Procedure for the Synthesis of Compound 17-20.

To a stirred solution of ethylpiperidine-4-carboxylate (1129 μL, 7.32 mmol) in anhydrous CH₂Cl₂ (3.5 mL) and triethylamine (1.53 mL, 10.98 mmol) ethyl chloroglyoxylate (1 g, 818 μL, 7.32 mmol) was added dropwise and very slowly at 0° C. under nitrogen gas atmosphere. The resulting yellow color mixture was further stirred at 0° C. under nitrogen gas atmosphere for 4 h. Cold water was added, and the reaction mixture was extracted with dichloromethane (2×20 mL). The combined organic layers were again washed with water, dried over anhydrous sodium sulfate and evaporated to dryness under reduced pressure. The desired diester 14 was isolated by flash chromatography (Combiflash, 12 g silica Redisep Rf Gold Cartridge, 25 mL/min flow rate, elution: 20-25% ethyl acetate in hexane) as colorless liquid (1.2 g, 63.8%).

Compound 14 (500 mg, 1.94 mmol) was taken in a mixture of THF (3 mL) and methanol (3 mL). To this aqueous NaOH (155 mg, 3.88 mmol in 4 mL H₂O) was added dropwise at 0 ° C. and stirred for 4 h under cold condition. THF and methanol were dried from the reaction mixture. The above solution was then diluted with water and extracted with ethyl acetate (20 mL). Aqueous layer was further acidified with 5% aqueous HCl (pH ˜5-6) and lyophilized. The dry mass was further washed with 30% methanol in dichloromethane (3×20 mL) and filtered through sintered funnel. Filtrate was then dried under reduced pressure, washed with diethyl ether (2×1 mL) and again dried to afford compound 18 as white solid (168 mg, 43%).

Compounds 17, 19, and 20 were prepared in a similar manner.

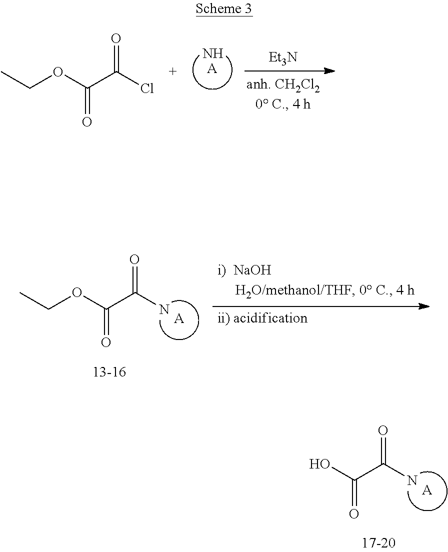

Ring A is a 4-7 membered heterocyclyl, wherein the 4-7 membered heterocyclyl is optionally substituted with one or more groups independently selected from halo, oxo, —CN, —NO₂, —CO₂H, —OH, (C₁-C₆)alkyl, (C₁-C₆)haloalkyl, —CO₂H, —O (C₁-C₆)alkyl, —O (C₁-C₆)haloalkyl and SO₂R^{a}.

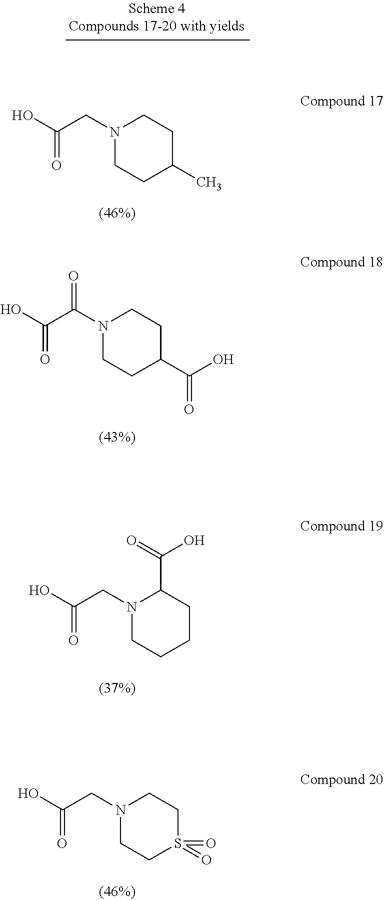

### Over-expression and Purification of SyrB1, SyrB2, Sfp, and TycF Proteins.

The sequence for SyrB1, SyrB2, and Sfp were synthesized in pET-28(a)+ vectors, which were generously provided from the Bollinger-Krebs Lab (Penn State). The sequence for TycF was synthesized in pET-30b vector. The following procedure was adapted from Matthews, et al. Biochemistry 48 (20), 4331-4343 (2009). BL21-Gold (DE3) competent cells (Agilent Technologies) were transformed with a specific plasmid and grown on LB agar plates spiked with kanamycin (100 μg/mL) overnight. For expression, all transformed cells were grown in 2XYT broth (16 g/L tryptone, 10 g/L yeast extract, 5 g/L NaCl 0.05 mg/L kanamycin, pH=7.0). A primary culture (50 mL) was grown overnight at 37° C. Equivalent aliquots of this primary culture were used to inoculate four 1.0 L secondary cultures in baffled 2.8 L cell culture flasks. These were grown at 37° C. till an OD₆₀₀=0.6-0.8 was achieved; after this, the cultures were cooled on ice for 15 min. and were induced with IPTG to a final concentration of 0.2 mM. Post-induction, the cultures were grown for 18 hrs at 18° C. TycF was expressed for 72 hr at 16° C. Cells were harvested by centrifugation (15 min, 8000 RPM, 4° C.). Cell pellets were flash frozen in liquid nitrogen and stored at −20° C. Typical pellet yields are 8-10 g/L of culture.

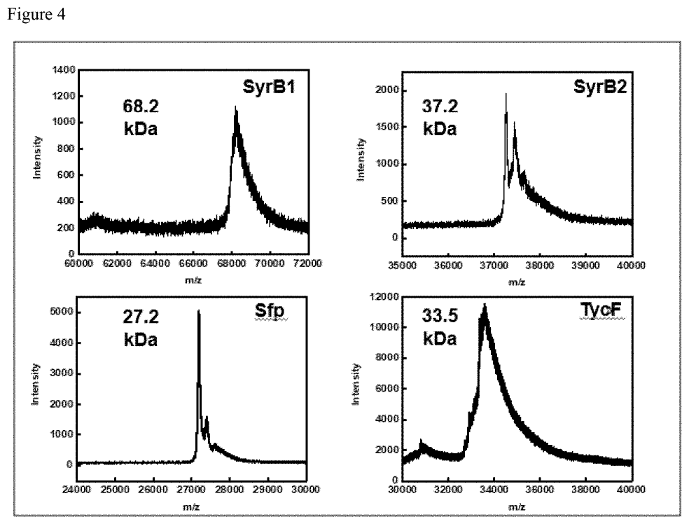

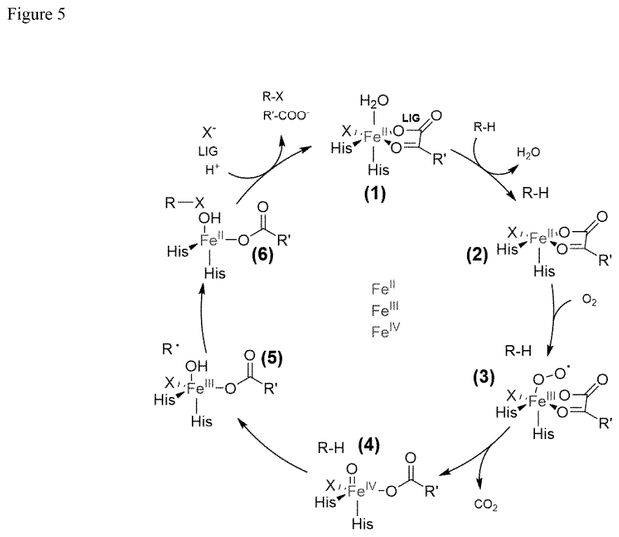

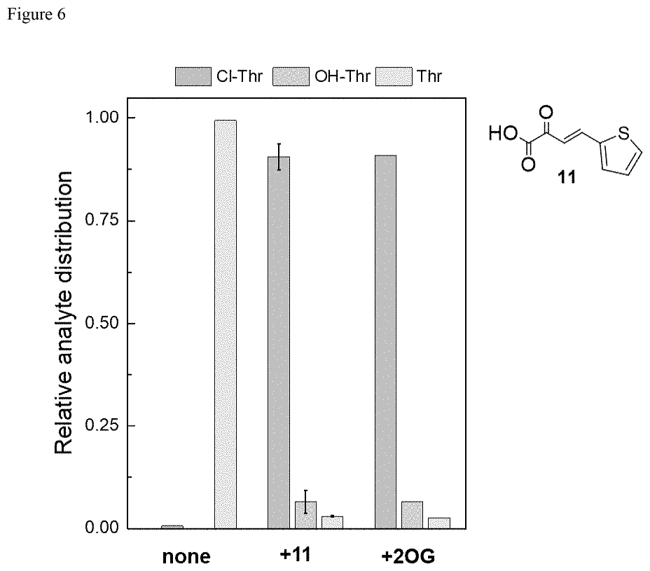

Cell pellets were resuspended (5 mL/g pellet) in Buffer A (50 mM HEPES, 300 mM NaCl, 5 mM imidazole, pH=7.5) supplemented with a Pierce Protease Inhibitor Tablet (Millipore-Sigma, 1 tablet/100 mL resuspension). Resuspension was sonicated using a program of: 30 s pulse-on, 30 s pulse-off. Cell lysate was centrifuged (20 min, 20000 RPM, 4° C.). The supernatant was collected, filtered (0.22 micron filter), and loaded onto a Ni HisTrap™ HP column (Cytiva) pre-equilibrated with Buffer A on an AKTA start purification system. The column was washed with at least 10 CV of Buffer A prior to elution. Protein was eluted by a gradient elution with Buffer B (20 mM Na-HEPES, 100 mM NaCl, 250 mM imidazole, pH=7.5). TycF was eluted using a stepwise isocratic gradient of 30% B to elute impurities and 100% B to elute TycF. Fractions corresponding to the relevant protein, as determined by SDS-PAGE gel electrophoresis, were pooled and dialyzed against 3 L of 20 mM HEPES buffer (pH=7.5) supplemented by 1 mM EDTA for at least 4 hr at 4° C. Two more dialyses against 3 L of 20 mM HEPES (pH=7.5) were performed for at least 4 hr each at 4° C. in order to remove any latent EDTA. Dialyzed samples were concentrated in an Amicon® Ultra Centrifugal Filters (molecular weight cutoff =50 kDa, 30 kDa, or 10 kDa for SyrB1, SyrB2, or Sfp/TycF, respectively) to a final concentrations of 1-4 mM. Protein concentrations were determined by the absorbance at 280 nm using molar extinction coefficients as determined by ProtParam on the ExPASy server. Protein was aliquoted in Axygen® Maxymum Recovery® Tubes flash frozen in liquid nitrogen, and stored at −80° C. The purity of expressed proteins were confirmed via protein gels and mass spectrometry (FIG. 4).

### Post-Translational Modifications of SyrB1: Phosphopantetheinylation and Subsequent Amino Acid Charging.

All described reactions occur in 20 mM HEPES buffer, pH=7.5 (RXN buffer). Apo-SyrB1 (100 μM) was incubated with the phosphopantetheinyl transferase Sfp (5 μM), 5 mM MgSO4, and 1 mM coenzyme A for 90 min. while stirring at room temperature. (Author tip: dissolve all substrates in RXN buffer and add them to the rxn vessel, adjust to near final volume, adjust the pH to 7.5, then add the proteins). The mixture was concentrated in a 50 kDa MWCO Amicon° Ultra Centrifugal Filter Unit, filtered, and loaded onto a HiLoad 26/600 Superdex 200 μg gel filtration column equilibrated with RXN buffer. SEC chromatography was implemented on an AKTA Pure instrument system (flow rate=0.8 mL/min, 3 injections). Fractions containing SyrB1-PPT, as determined by SDS-PAGE, were pooled, concentrated as described above, and taken for the subsequent AA charging rxn.

SyrB1-PPT (100 μM) was incubated with MgSO₄ (5 mM), ATP (10 mM), and relevant amino acid (10 mM L-threonine) in RXN buffer. The solution was stirred at room temperature for 30 minutes, before transfer to ice. (Author Note: It's crucial for any AA charged sample to remain cool from this point onward to prevent spontaneous thioester hydrolysis of the charged amino acid). The reaction was concentrated and buffer exchanged with cold RXN buffer three times to remove any unreacted substrates. The subsequent product was aliquoted into Axygen° Maxymum Recovery® Tubes, flash frozen in liquid nitrogen, and stored at −80° C.

### Fluorination Assays.

In an anaerobic glovebag (98% N₂/2% H₂, Coy Laboratories), all plasticware, solutions, and solids were allowed to equilibrate overnight before use. Stock solutions of RXN buffer (20 mM HEPES, pH=7.5), 20G (100 mM in RXN buffer, pH=7.5), NOG (150 mM in RXN buffer, pH=7.5), and Milli-Q water were degassed using a vacuum manifold on a Schlenk Line apparatus. At least three cycles of vacuum and subsequent equilibration with Argon gas were performed before transfer to the glovebag. Halide Salts (NaCl, NaF) and ferrous ammonium sulfate (FAS) were transferred to the glovebag the day before use.

Stock SyrB1-PPT-Thr and SyrB2 were thawed on ice-water and centrifuged to remove any possible precipitate. Stock proteins were diluted in RXN buffer and transferred to individual ReactiVials (Thermo) sealed with septa on a pre-cooled ReactiBlock (Thermo). Protein was deoxygenated by piercing the ReactiVials with needles and purging the headspace with a continuous flow of Argon gas for 1 hr while continuously stirring the protein solution on ice bath. Reactivials were sealed and transferred to the glovebag. While deoxygenating the protein, halide salts were dissolved in RXN buffer in the glovebag. To individual 1.7 mL tubes, RXN buffer, ligand (NOG or 2OG), and halide (NaCl or NaF) were added and cooled to 4° C. The ReactiVials containing the proteins were sealed and transferred to the glovebag. Immediately upon transfer, the protein solutions were aliquoted into pre-cooled tubes and centrifuged to remove any precipitate (all centrifugation steps were performed at 14000*g for 5 min. at 4° C., unless otherwise stated). To each reaction tube, the appropriate amount of SyrB2 was added. After SyrB2 addition, the FAS salt was dissolved in MilliQ water, and the appropriate amount was added to the reaction tube, which was then centrifuged. SyrB 1-PPT-Thr was then added to each reaction tube, mixed by pipetting, and then all tubes were centrifuged for the final time. After addition of substrate, the final concentrations for all species are as follows: [SyrB1-PPT-Thr]=[SyrB2]=[FAS]=100 uM, [2OG or NOG]=1 mM, [NaCl or NaF]=10 mM. Reaction volumes were 200 μL.

Reactions were Performed According to One of the Following Methods.

All sealed reaction tubes were brought outside the glovebag and spiked with internal standard (¹³C₄-Thr) to 500 nM concentration. Holes were punched in the tops of the tubes to facilitate the transfer of gases in solution. Reaction tubes were left on a Thermomixer (25° C., 300 RPM) for 12 hr. Overnight incubation hydrolyzes the thioester bond between the threonine and the PPT arm in the SyrB1-PPT-Thr substrate. The tubes were then centrifuged to remove any precipitate and the supernatant was transferred to pre-washed 0.5 mL 10 kDa MWCO Amicon® Ultra Centrifugal Filter Units (washed with 500 μL of 100 mM NaOH then 500 μL of RXN buffer) and concentrated to isolate the small-molecule containing flow-through from the protein. Flow-through was transferred to deactivated, silanized glass vials (Waters Corp.), flash frozen in liquid nitrogen, and stored at −80° C. All samples were then lyophilized prior to amino acid derivatization and UHPLC/MS-MS analysis.

All sealed reaction tubes were brought outside the glovebag and put on ice bath. To each reaction tube, 200 μL of room-temperature O₂ saturated RXN buffer was spiked and the now 400 μL solution was mixed by pipetting. All reaction solutions were incubated on a Thermomixer (25° C., 300 RPM) for 10 min. Reaction solutions were transferred to pre-washed 0.5 mL 10 kDa MWCO Amicon® Ultra Centrifugal Filter Units (washed with 500 μL of 100 mM NaOH then 500 uL of RXN buffer), and concentrated to —50 μL. The centricons were then filled to 400 μL with RXN buffer and concentrated again. This process was repeated two more times to remove any other possible small molecule interferants or excess amino acid. Reaction volumes were then diluted to 125 μL and spiked with TycF thioesterase to a final concentration of 5 μM. This thioesterase liberates threonine (and associated products) from the thioester bond linking the amino acid to the SyrB1-PPT-Thr substrate. After 90 min. of incubation at 25° C., the solutions were diluted to 400 μL with reaction buffer and concentrated to ˜50 μL. This dilution/concentration process was repeated 3 more times to fully isolate the product-containing flow-through. Flow through was spiked with internal standard (13C4-Thr) to 500 nM concentration. Flow-through was transferred to deactivated, silanized glass vials (Waters Corp.), flash frozen in liquid nitrogen, and stored at −80° C. All samples were then lyophilized prior to amino acid derivatization and UHPLC/MS-MS analysis.

Method for Chlorination Reaction with SyrB2

SyrB2 (50 μM) was added to a solution of 50 μM SyrB1-Ppt-Thr, 50 μM ferrous ammonium sulfate, 50 mM NaCl, and 500 μM 2OG or 2OG analog in 20 mM HEPES (pH=7.5) with a final reaction volume of 200 μL. Reaction tubes were transferred to an Eppendorf ThermoMixer and incubated for an hour at 25° C. and 300 RPM. After incubation, reactions were concentrated in pre-washed (100 mM NaOH, then 20 mM HEPES (pH=7.5)) 0.5 mL Amicon

Ultra 10 kDa MWCO centrifugal filter units at 4° C. Once initially concentrated, reaction solutions were washed 3 times by diluting the volume to 500 μL and then concentrating again. To cleave functionalized products from carrier protein SyrB1, reaction samples were diluted to a final volume of 150 μL with 20 mM HEPES (pH=7.5) in the centricons and incubated with TycF thioesterase (5 μM) for 90 min at 25° C. Reaction samples were then again diluted to 400 μL with 20 mM HEPES (pH=7.5) in the centricons, concentrated, and then flow-through was collected into deactivated, silanized Waters vials. This washing step was repeated two more times with flow-through being collected each time to recover all cleaved products. The collected solution (˜1.2 mL) was then lyophilized overnight. The dry product was reconstituted in 160 μL of 10 mM borate buffer and 20 μL of 375 mM NaOH was added to each solution to bring the final pH to 8.5. After reconstitution, 20 ul of 60 mM 6-aminoquinolyl-N-hydroxysuccinimidyl carbamate in acetonitrile was added to each sample and then, immediately after this addition, samples were mixed by vortexing for 15 seconds to ensure proper derivatization. To analyze derivatized reaction products, samples were run on a Waters Acquity UPLC coupled to a Waters triple quadrupole mass spectrometer (Acquity TQD) as previously described in Wilson et. al. ACS Catalysis 2022.

All publications, patents, and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

What is claimed is:

1. A method to convert a —CH- group of a substrate compound or a salt thereof, to a corresponding —CX- group of a product compound or a salt thereof, comprising contacting the substrate compound or a salt thereof with a nonheme iron halogenase enzyme and a ligand activator, wherein:
the carbon atom of the —CH- group and the —CX- group is sp³ hybridized;
X is halo, —N₃, —NO₂, —CN, or —SR; and
R is H or (C₁-C₈)alkyl.

2. The method of claim 1, wherein the ligand activator comprises two or more oxygen atoms, wherein at least two of the oxygen atoms coordinate to the iron of the nonheme iron enzyme.

3. The method of claim 1, wherein the ligand activator comprises one or more oxo (═O) groups.

4. The method of claim 1, wherein the ligand activator comprises two or more oxo (═O) groups.

5. The method of claim 1, wherein the ligand activator comprises one or more hydroxy groups.

6. The method of claim 1, wherein the ligand activator comprises two oxo (═O) groups and one hydroxy group.

7. The method of claim 1, wherein the ligand activator comprises one or more groups of formula IV:

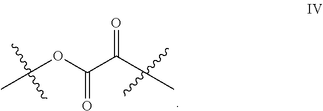 [SMILES: *OC(=O)C(*)=O]

8. The method of claim 1, wherein the ligand activator comprises one or more groups of formula IV:

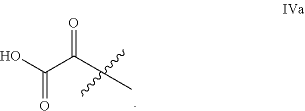 [SMILES: *C(=O)C(=O)O]

9. The method of claim 1, wherein the ligand activator is a compound of formula I, formula II or formula III: 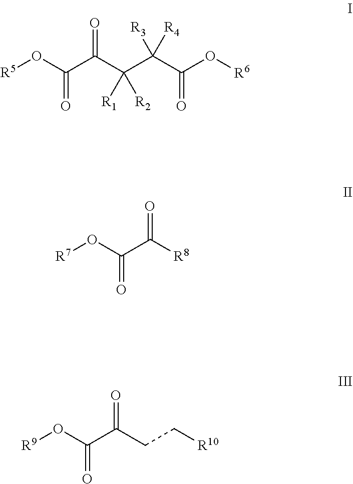
wherein:
the dashed bond of the compound of formula III is a single bond or a double bond;
R¹, R², R³, R⁴, are each independently hydrogen, halo, (C₁-C₈)alkyl, —CO₂H, —OH, 5-6 membered heteroaryl, 4-7 membered heterocyclyl, or phenyl, wherein the (C₁-C₈)alkyl, 5-6 membered heteroaryl, 4-7 membered heterocyclyl, or phenyl is optionally substituted with one or more groups independently selected from halo, oxo, —CN, —NO₂, —CO₂H, —OH, (C₁-C₆)alkyl, (C₁-C₆)haloalkyl, —O (C₁-C₆)alkyl, —O (C₁-C₆)haloalkyl and SO₂R^{a};
R⁵ is hydrogen or (C₁-C₈)alkyl;
R⁶ is hydrogen, (C₁-C₈)alkyl, or phenyl, wherein phenyl is optionally substituted with one or more groups independently selected from halo, oxo, —CN, —NO₂, —CO₂H, —OH, (C₁-C₆)alkyl, (C₁-C₆)haloalkyl, —O (C₁-C₆)alkyl, —O (C₁-C₆)haloalkyl and SO₂R^{a};
R⁷ is hydrogen or (C₁-C₈)alkyl;
R⁸ is (C₃-C₇)carbocyclyl, 4-7 membered heterocyclyl, phenyl, OR^{b}, SR^{b}, -(C₁-C₆)alkylOR^{b}, or —(C₁-C₆)alkylSR^{b} wherein the (C₃-C₇)carbocyclyl, 4-7 membered heterocyclyl, phenyl, -(C₁-C₆)alkylOR^{b}, or -(C₁-C₆)alkylSR^{b}, is optionally substituted with one or more groups independently selected from halo, oxo, —CN, —NO₂, —CO₂H, —OH, (C₁-C₆)alkyl, (C₁-C₆)haloalkyl, —CO₂H, —O (C₁-C₆)alkyl, —O (C₁-C₆)haloalkyl and SO₂R^{a};
R⁹ is hydrogen or (C₁-C₈)alkyl;
R¹⁰ is -(C₁-C₁₀)alkyl, -(C₁-C₁₀)alkenyl, R^{10a}, -(C₁-C₁₀)alkylR^{10a}, or -(C₁-C₁₀)alkenylR^{10a}, wherein the -(C₁-C₁₀)alkyl, -(C₁-C₁₀)alkenyl, -(C₁-C₁₀)alkylR^{10a}, or -(C₁-C₁₀)alkenylR^{10a} is optionally substituted with one or more halo;
each R^{11a} is independently aryl or heteroaryl, wherein the aryl or heteroaryl is optionally substituted with one or more groups independently selected from halo, —CN, —NO₂, —CO₂H, —OH, (C₁-C₆)alkyl, (C₁-C₆)haloalkyl, —O (C₁-C₆)alkyl, —O (C₁-C₆)haloalkyl, and SO₂R^{a};
R^{a} is —OH, (C₁-C₆)alkyl, or (C₁-C₆)haloalkyl;
R^{b} and RC are each independently (C₁-C₈)alky, wherein (C₁-C₈)alky is optionally substituted with one or more groups independently selected from halo, CO₂H, oxo, —OH, 5-6-membered heteroaryl and —N═C(NR^{d}₂)₂, and wherein 5-6-membered heteroaryl is optionally substituted with one or more groups independently selected from halo, oxo, —CN, —NO₂, —CO₂H, —OH, (C₁-C₆)alkyl, (C₁-C₆)haloalkyl, —O (C₁-C₆)alkyl, —O (C₁-C₆)haloalkyl, and SO₂R^{a}; and
each R^{d} is independently hydrogen or (C₁-C₆)alky;
or a salt thereof.

10. The method of claim 9, wherein the ligand activator is a compound of formula I, formula II or formula III: 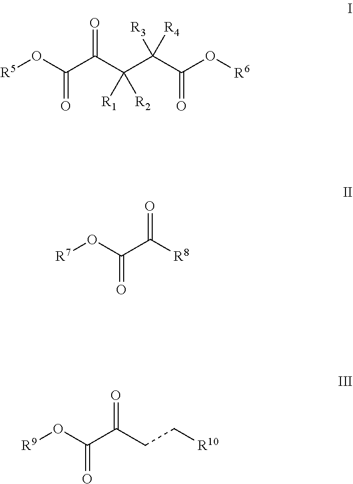
wherein:
the dashed bond of the compound of formula III is a single bond or a double bond;
R¹, R², R³, R⁴, are each independently hydrogen, halo, (C₁-C₈)alkyl, —CO₂H, —OH, 5-6 membered heteroaryl, 4-7 membered heterocyclyl, or phenyl, wherein the (C₁-C₈)alkyl, 5-6 membered heteroaryl, 4-7 membered heterocyclyl, or phenyl is optionally substituted with one or more groups independently selected from halo, oxo, —CN, —NO₂, —CO₂H, —OH, (C₁-C₆)alkyl, (C₁-C₆)haloalkyl, —O (C₁-C₆)alkyl, —O (C₁-C₆)haloalkyl and SO₂R^{a};
R⁵ is hydrogen or (C₁-C₈)alkyl;
R⁶ is hydrogen, (C₁-C₈)alkyl, or phenyl, wherein phenyl is optionally substituted with one or more groups independently selected from halo, oxo, —CN, —NO₂, —CO₂H, —OH, (C₁-C₆)alkyl, (C₁-C₆)haloalkyl, —O (C₁-C₆)alkyl, —O (C₁-C₆)haloalkyl and SO₂R^{a};
R⁷ is hydrogen or (C₁-C₈)alkyl;
R⁸ is (C₃-C₇)carbocyclyl, 4-7 membered heterocyclyl, phenyl, OR^{b}, SR^{b}, -(C₁-C₆)alkylOR^{b}, or -(C₁-C₆)alkylSR^{b} wherein the (C₃-C₇)carbocyclyl, 4-7 membered heterocyclyl, phenyl, -(C₁-C₆)alkylOR^{b}, or -(C₁-C₆)alkylSR^{b}, is optionally substituted with one or more groups independently selected from halo, oxo, —CN, —NO₂, —CO₂H, —OH, (C₁-C₆)alkyl, (C₁-C₆)haloalkyl, —CO₂H, —O (C₁-C₆)alkyl, —O (C₁-C₆)haloalkyl and SO₂R^{a};
R⁹ is hydrogen or (C₁-C₈)alkyl;
R¹⁰ is -(C₁-C₁₀)alkenyl, R^{10a}, -(C₁-C₁₀)alkylR^{10a}, or -(C₁-C₁₀)alkenylR¹⁰a, wherein the -(C₁-C₁₀)alkyl, -(C₁-C₁₀)alkenyl, -(C₁-C₁₀)alkylR^{10a}, or -(C₁-C₁₀)alkenylR^{10a} is optionally substituted with one or more halo;
each R^{10a} is independently phenyl, wherein the phenyl is optionally substituted with one or more groups independently selected from halo, —CN, —NO₂, —CO₂H, —OH, (C₁-C₆)alkyl, (C₁-C₆)haloalkyl, —O (C₁-C₆)alkyl, —O (C₁-C₆)haloalkyl, and SO₂R^{a};
R^{a} is —OH, (C₁-C₆)alkyl, or (C₁-C₆)haloalkyl;
R^{b} and RC are each independently (C₁-C₈)alky, wherein (C₁-C₈)alky is optionally substituted with one or more groups independently selected from halo, CO₂H, oxo, —OH, 5-6-membered heteroaryl and —N═C(NR^{d}₂)₂, and wherein 5-6-membered heteroaryl is optionally substituted with one or more groups independently selected from halo, oxo, —CN, —NO₂, —CO₂H, —OH, (C₁-C₆)alkyl, (C₁-C₆)haloalkyl, —O (C₁-C₆)alkyl, —O (C₁-C₆)haloalkyl, and SO₂R^{a}; and
each R^{d} is independently hydrogen or (C₁-C₆)alky;
or a salt thereof.

11. The method of claim 9, wherein the ligand activator is a compound of formula III: 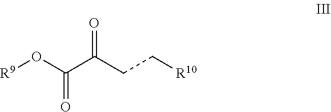
or a salt thereof.

12. The method of claim 9, wherein the ligand activator is a compound of formula: 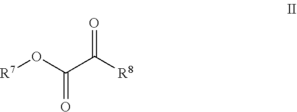
or a salt thereof

13. The method of claim 9, wherein the ligand activator is: 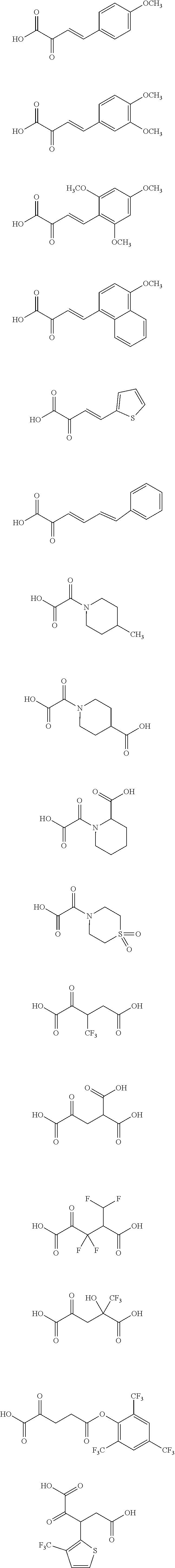 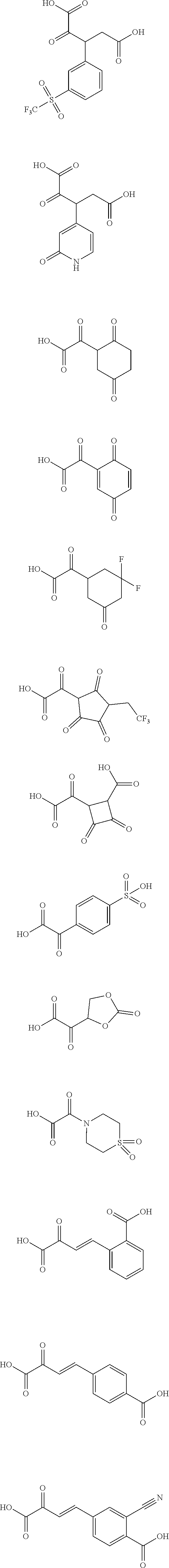 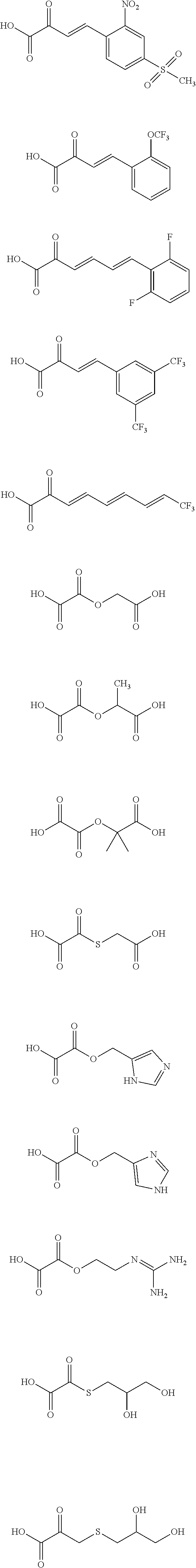
or an ester(s) or a salt thereof.

14. The method of claim 1, wherein the ligand activator is 2-oxoglutaric acid or N-oxalylglycine or a salt thereof.

15. The method of claim 1, wherein X is halo.

16. The method of claim 1, wherein the nonheme iron halogenase enzyme is SyrB2, KthP, CytC3, BarB1/BarB2, KtzD, CurA, CmaB, HctB, WelO5, AmbO5, AdeV, BesD, HalB, HalC, HalD or HalE.

17. The method of claim 1, further comprising contacting the substrate compound with a reagent comprising X.

18. The method of claim 17, wherein the reagent comprises F or Cl.

19. The method of claim 18, wherein the reagent comprising F is sodium fluoride, potassium fluoride, tetrabutylammonium fluoride, tetrabutylammonium fluoride with silver fluoride and/or cesium fluoride, diethylaminosulfur trifluoride (DAST), (diethylamino)difluorosulfonium tetrafluoroborate (XtalFluor-E), N-Fluoro-N′-methyl-triethylenediamine bis(tetrafluoroborate) (Selectfluor II) or N-Fluorobenzenesulfonimide (NFSI).

20. The method of claim 1, wherein substrate compound is an amino acid, nucleotide, natural product or synthetic compound.
